(19) United States
(12) Patent Application Publication
(10) Pub. No.: US 2024/0149005 A1
(43) Pub. Date: May 9, 2024

(54) HME DEVICES

(71) Applicant: SMITHS MEDICAL INTERNATIONAL LIMITED, Ashford (GB)

(72) Inventors: Usamah Hanif, Southend-on-Sea (GB); Christopher John Woosnam, London (GB); Timothy Bateman, Hythe (GB); Steven Mark Tupper, Hythe (GB)

(73) Assignee: SMITHS MEDICAL INTERNATIONAL LIMITED, Ashford (GB)

(21) Appl. No.: 18/281,701

(22) PCT Filed: Apr. 4, 2022
(86) PCT No.: PCT/GB2022/000047

§ 371 Date: Sep. 12, 2023

(30) Foreign Application Priority Data
Apr. 12, 2021 (GB) 2105159.4

(51) Int. Cl. A61M 16/10; A61M 16/08

(52) U.S. Cl. CPC A61M 16/1045; A61M 16/0816

(57) ABSTRACT

An HME device (1) has an outer housing (10) in two parts removably connected with one another by bayonet lugs (17) on one part (13) inserted in bayonet slots (48) on the other part (14) and twisted. An HME unit (50) has a tapered coupling at one end fitted on an internal port (45) in one part (14) so that it can be removed and replaced by separating the two parts of the housing. The outside of the HME unit (50) is spaced from the inside of the housing (10) by an annular bypass channel (55). A selector (31) has a rotatable plate that can be moved to cover either the passage through the HME unit or the bypass channel.

This invention relates to HME devices.

Where a patient breathes through a tube inserted in the trachea, such as a tracheostomy or endotracheal tube, gas flow to the bronchi is not warmed and moistened by passage through the nose. Unless the gas is warmed and moistened in some way it can cause damage and discomfort in the patient's throat. The gas can be conditioned by a humidifier in the ventilation circuit but, most conveniently, a heat and moisture exchange device (HME) is used. HMEs are small, lightweight devices including one or more exchange elements, such as of a paper or foam treated with a hygroscopic substance. When the patient exhales, gas passes through the exchange element and gives up a major part of its heat and moisture to the element. When the patient inhales, gas passes through the exchange element in the opposite direction and takes up a major part of the heat and moisture in the exchange element so that the gas inhaled by the patient is warmed and moistened. These HMEs are low cost and disposable after a single use. They can be connected in a breathing circuit or simply connected to the machine end of a tracheal tube and left open to atmosphere where the patient is breathing spontaneously. HMEs can be used with other breathing devices such as face masks.

HMEs are sold by Smiths Medical International Limited of Ashford, Kent, England under the Thermovent name (Thermovent is a registered trade mark of Smiths Medical International Limited), by Hudson RCI AB under the TrachVent name (TrachVent is a registered trade mark of Hudson RCI AB), by DAR, Medisize, Intersurgical and other manufacturers. Examples of HMEs are described in GB2391816, WO01/72365, U.S. Pat. No. 5,505,768, SE516666, U.S. Pat. No. 3,881,482, DE20302580, DE20114355U, WO97/01366, US2002/0157667, U.S. Pat. No. 6,422,235, EP1208866, U.S. Pat. No. 4,971,054, EP1699515, U.S. Pat. No. 5,035,236, EP535016, U.S. Pat. No. 5,647,344, GB2267840, EP856327, EP1699515, U.S. Pat. No. 7,363,925, WO15/107320, US2008/0099013, GB2540456 and WO2017/216508. The “Thermovent T” HME sold by Smiths Medical has a T-shape configuration with two HME elements mounted at opposite ends of a straight tubular housing extending transversely of the connection port by which the device is fitted onto a tracheostomy tube or the like. The tubular housing for the HME elements may be curved to follow the anatomical profile of the neck, as described in EP1888157.

HMEs are often used in breathing circuits for patients in intensive care where it may be necessary to be able to administer drugs by nebulisation into the lungs. It is not advisable to administer nebulised drugs via the HME media because this may interact or capture the nebulised drugs and reduce the amount reaching the lungs. It would also reduce the efficiency of the HME. In such circumstances the breathing circuit has to be disconnected to remove the HME and connect the nebuliser, leaving the patient without ventilation for a time. After the drugs have been administered the HME is reconnected in the breathing circuit, which, once again, means that the patient is without ventilation for a time. This can be detrimental to the patient and takes time for the clinician. To avoid this a by-pass system could be used where an additional length of tubing is connected into the breathing system by a two-way valve and extends around the HME so that gas can flow through this tubing instead of through the HME when drugs need to be administered. These systems, however, tend to be bulky, cumbersome and add weight and dead space to the breathing circuit. The bypass tubing can also snag on adjacent equipment.

It is an object of the present invention to provide an alternative HME device.

According to the present invention there is provided an HME device, characterised in that the device can be selectively set in an HME mode or a bypass mode, that the device includes an outer housing having at least two parts that can be manually connected or disconnected from one another to enable access to the interior of the housing, that one of the parts has an external port by which gas can flow through the housing and an internal port in selective flow communication with the external port, that the device includes an HME unit arranged to be manually connected and disconnected with the internal port such that the two parts of the housing can be separated from one another to enable removal and replacement of the HME unit on the internal port, that the exterior of the HME unit and the interior of the outer housing are configured to provide a bypass channel between them to enable gas flow through the device along the bypass channel when in the bypass mode, that the device includes a selector that in a bypass position selectively blocks gas flow through the internal port so that gas flow through the device bypasses the HME unit and flows instead along the bypass channel, and that the selector in an HME position enables flow through the device via the HME unit and blocks flow along the bypass channel.

The two parts of the outer housing may be connected with each other by bayonet lugs on one part and slots on the other part that are arranged to receive the lugs when the two parts are twisted relative to each other. The bypass channel is preferably provided by an annular passage between the outside of the HME unit and the inside of the outer housing. The HME unit preferably has a tapered coupling at one end removably fitted on a tapered fitting in one part of the outer housing. The selector may include a shutter plate movable in a plane at right angles to the axis of the device between a position where it covers an opening to the HME unit and reveals a bypass opening to a position where it reveals the opening to the HME unit and covers the bypass opening.

An HME device according to the present invention will now be described, by way of example, with reference to the accompanying drawings, in which:

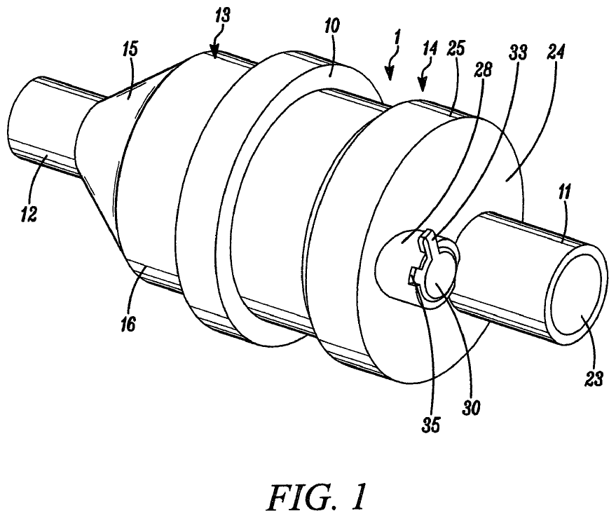

FIG. 1 is a perspective view of the HME device from its patient end;

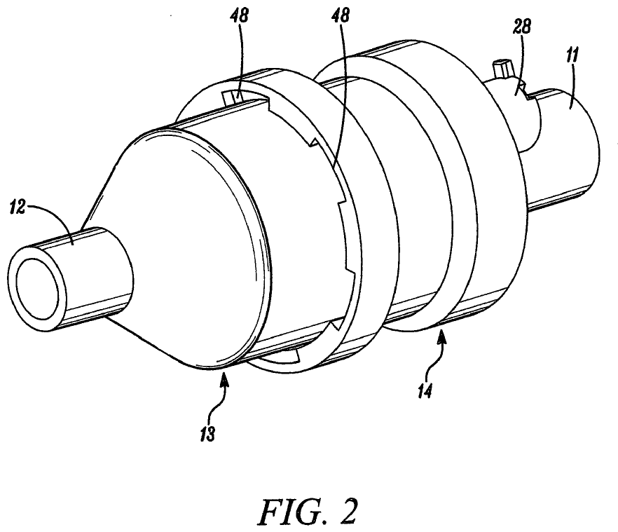

FIG. 2 is perspective view of the HME device from its machine end;

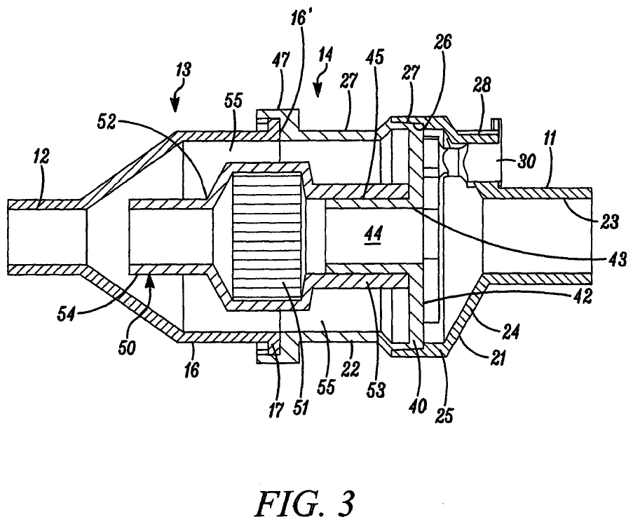

FIG. 3 is a cross-sectional side elevation view of the HME;

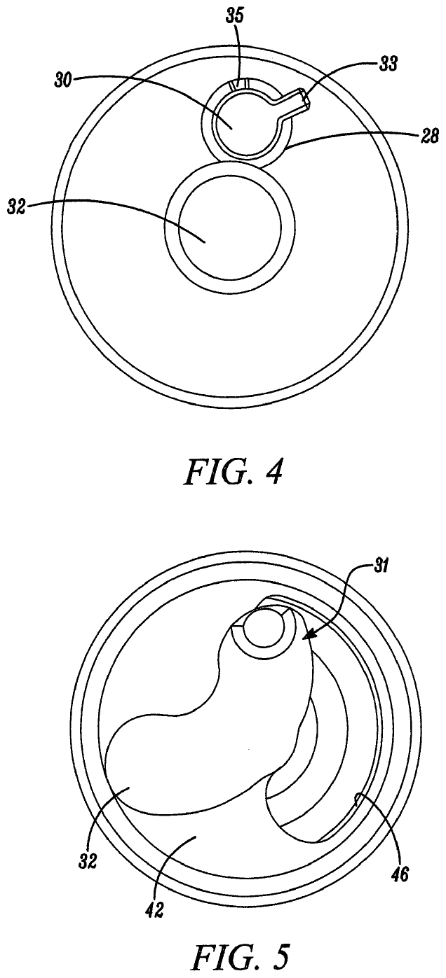

FIG. 4 is an end view from the patient end of the HME device in the bypass mode;

FIG. 5 is an end view of the interior of the device in the bypass mode;

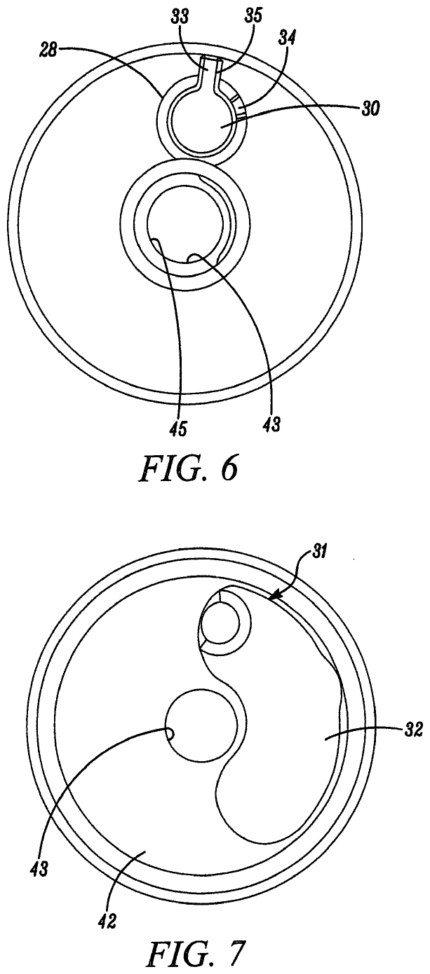

FIG. 6 is an end view from the patient end in the HME mode;

FIG. 7 is an end View of the interior of the device in the HME mode;

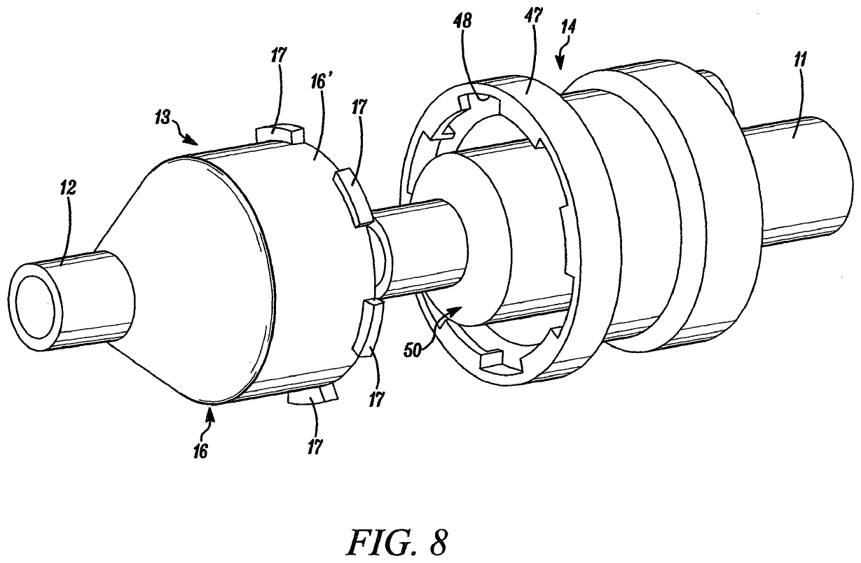

FIG. 8 is a perspective view of the device from the machine end showing the two parts of the outer housing released and separated; and

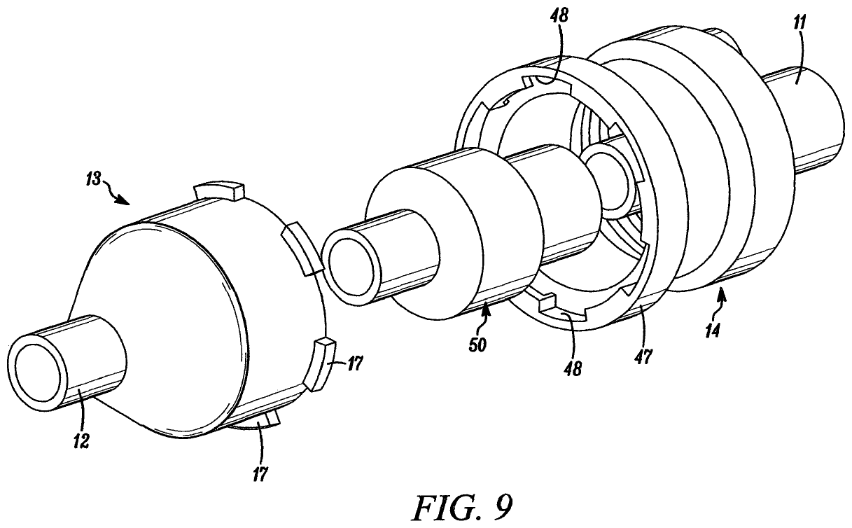

FIG. 9 is a perspective view of the device with the two parts of the housing separated and with the HME unit uncoupled for removal and replacement.

With reference first to FIGS. 1 to 3 there is shown an HME device 1 having an outer housing 10 of circular section with a patient end coupling or inlet 11 adapted to fit onto a mating male connector at the end of a breathing device (not shown) such as an endotracheal or tracheostomy tube. At the opposite end of the device 1 a machine end coupling or outlet 12 is axially aligned with the patient end coupling 11. Between the two couplings 11 and 12 the housing is radially enlarged and of circular section. The outer housing 10 has two parts 13 and 14 that are connected with each other by a releasable rotatable coupling arrangement so that they can be separated to enable access to the interior of the housing. More particularly, the left-hand, machine end part 13 includes the machine end coupling 12, a mid-section 15 that tapers to an enlarged end section 16 of cylindrical shape. At its free end 16′ the cylindrical section 16 has six outwardly projecting bayonet lugs 17 spaced equally around the cylindrical section, as shown most clearly in FIGS. 8 and 9.

The other part of the outer housing 10 is provided by the right-hand, patient end part 14, which comprises two moulded components 21 and 22 bonded together end-to-end. The right-hand, patient end component 21 includes the patient end coupling 11, which has an internal, tapered surface 23 adapted for mating connection with a standard 15 mm male coupling on the end of a tracheal tube or other breathing device. The patient end component 21 also has a mid-section 24 that tapers outwardly to an enlarged cylindrical collar 25. The collar 25 has a tapered internal surface 26 at its end bonded to a cooperating tapered surface 27 at the right-hand, patient end of the other component 22. The tapered mid-section 24 of the patient end component 21 is also formed with a short, outwardly projecting small diameter sleeve 28 that supports within it a knob 30 of a mode selector 31. With reference now also to FIGS. 5 and 7, the mode selector 31 has a curved, planar shutter plate 32 of C shape the plane of which extends at right angles to the axis of the HME device 1. The knob 30 is fixedly attached with one end of the plate 32 so that when the knob is twisted in the sleeve 28 it rotates the plate about the axis of the knob. The knob 30 has a short locking finger 33 projecting radially outwardly that can be located in one of two notches 34 or 35 in the outer end of the sleeve 28, the two notches being spaced from one another by about 60°. Operation of the mode selector 31 will be described in more detail later.

The component 22 at the machine end of the patient end part 14 has a slightly enlarged rim 40 at its patient end, with the tapered external surface 27 fitting within and being bonded to the inside of the collar 25 on the patient end component 21. A wall 42 extends laterally across the patient end of the component 22 as an integral moulding. The wall 42 has a central circular opening 43, which communicates with a bore 44 extending through an axial tubular formation or port 0.45. The formation 45 extends coaxially of the housing 10 towards its machine end, about half way along the length of the patient end part 14. This tubular formation 45 is tapered on its external surface and provides an internal port of the same shape and dimensions as a standard 15 mm male coupling. The wall 42 also has a second opening through it provided by a curved slot 46 (FIG. 5) towards the outer edge of the wall, extending through about 120° and centred on the axis of the device. The dimensions of the slot 46 are slightly less than those of the shutter plate 32 so that, when the shutter plate is displaced to its full extent anticlockwise it completely covers the slot, as shown in FIG. 7.

The opposite, machine end of the component 22 is formed with a radially-enlarged rim 47 adapted to receive the patient end 16′ of the machine end part 13. The end face of the rim 47 is interrupted with six L-shape slots 48. The size and shape of the slots 48 are selected to enable the lugs 17 on the machine end part 13 to be inserted and rotated through about 30° to lock the two parts 13 and 14 together in a substantially gas-tight manner, thereby providing a rotatable coupling between the two parts.

As shown in FIG. 3, the device 1 includes a conventional HME unit 50 of the kind having an HME element 51 contained within an outer plastics housing 52 with a patient end coupling 53 at one end. The patient end coupling 53 has an internal tapered surface providing a female 15 mm fitting adapted to be fitted on a 15 mm tapered male fitting provided by the internal port 45. At its opposite end the HME unit 50 has a machine end fitting 54 with an external tapered surface adapted to provide a 15 mm male fitting. In the present example the machine end fitting 54 of the HME unit 50 opens into the interior of the outer housing 10 and is not connected to any other component. The end fittings 53 and 54 are axially aligned with each other with the HME element 51 being positioned between them so that all gas flow through the HME unit 50 in either direction passes through the HME element. The HME element 51 may be of a conventional kind such as including a coil of corrugated paper treated with a hygroscopic salt. Alternatively, it could be a foam element. The HME could also include a filter. The maximum external diameter of the HME unit 50 is less than the internal diameter of the outer housing 10 so that there is bypass channel in the form of an annular passage 55 extending along and around the outside of the HME unit, between the outside of the HME unit and the inside of the outer housing 10.

The HME device 1 can be set to either an HME mode, during normal use, or to a bypass mode when nebulisation or drugs need to be delivered to the airway. In the bypass mode the gas is prevented from flowing through the HME unit 50 because this would absorb the nebulised drug and reduce the amount passing to the patient; it would also reduce the subsequent effectiveness of the HME. The device 1 is switched between these two modes using the knob 30 to move the shutter plate 32 in its plane, at right angles to the axis of the device 1. In the state shown in FIGS. 6 and 7 the knob 30 is in its furthest position anticlockwise with its locking finger 33 located in the anticlockwise notch 35. In this position the shutter plate 32 is swung anticlockwise to its full extent in engagement with the inside of the collar 25. The shutter plate 32 therefore covers the bypass opening 46 and reveals the central opening 43 to the internal port 45 and hence to the HME unit 50. In this mode, therefore, all the gas flowing between the two couplings 11 and 12 of the device flows through the HME unit 50.

If the clinician needs to administer a nebulised drug, he lifts the locking finger 33 on the knob 30 out of the HME notch 35 and twists the knob clockwise to its maximum extent (about 60°) until the locking finger aligns with and snaps into the bypass notch 34 as shown in FIG. 4. FIG. 5 shows that, in this position, the shutter plate 32 is swung to its full extent clockwise so that the central opening 43 to the HME is closed. The shutter plate 32 is also moved away from the bypass opening 46 so that most of this is uncovered. Gas can then flow between the end couplings 11 and 12, through the bypass opening 46 and along the bypass channel 55 around the outside of the HME unit 50 without flowing through the HME unit. When medication delivery ceases the clinician simply twists the selector knob 30 back to its HME mode position so that gas again flows backwards and forwards through the HME unit 50.

Conventional HMEs need to be replaced approximately every 24 hours to avoid the accumulation of bacteria in the HME element and to maintain maximum efficiency. In the present arrangement this is carried out by simply twisting the machine end part 13 of the outer housing 10 through about 30° relative to the patient end part 14 of the outer housing 10 so that the bayonet lugs 17 on the machine end part disengage the L-shape slots 48 on the patient end part, thereby enabling the two parts of the outer housing to be separated from one another in the manner shown in FIG. 8. This can be achieved without the need to disconnect the fittings 11 and 12 from the breathing circuit. It can be seen that, in this state, the machine end of the HME unit 50 projects beyond the machine end of the patient end part 14 of the housing 10 so that it can be easily gripped and twisted relative to the patient end part to disconnect the patient end of the HME unit from the internal port 45 in the manner shown in FIG. 9. In this way, the HME unit 50 can be removed from the outer housing 10, disposed of and replaced by a new HME unit. After this, the machine end part 13 of the outer housing 10 is reconnected with the patient end part 14 of the housing.

The arrangement of the present invention enables a bypass to be provided for an HME in a compact arrangement without external tubing that could become tangled or snag on adjacent equipment. The present invention also enables a major part of the device to be reused with several different HMEs. This saves expense and reduces the amount of plastics waste, which is a particular advantage in medical devices because the cost of disposing of clinical waste is greater than conventional waste.

1-5. (canceled)

6. An HME device, characterised in that the device can be selectively set in an HME mode or a bypass mode, that the device includes an outer housing having at least two parts that can be manually connected or disconnected from one another to enable access to the interior of the housing, that one of the parts has an external port by which gas can flow through the housing and an internal port in selective flow communication with the external port, that the device includes an HME unit arranged to be manually connected and disconnected with the internal port such that the two parts of the housing can be separated from one another to enable removal and replacement of the HME unit on the internal port, that the exterior of the HME unit and the interior of the outer housing are configured to provide a bypass channel between them to enable gas flow through the device along the bypass channel when in the bypass mode, that the device includes a selector that in a bypass position selectively blocks gas flow through the internal port so that gas flow through the device bypasses the HME unit and flows instead along the bypass channel, and that the selector in an HME position enables flow through the device via the HME unit and blocks flow along the bypass channel.

7. An HME device according to claim 6, characterised in that the two parts of the outer housing are connected with each other by bayonet lugs on one part and slots on the other part that are arranged to receive the lugs when the two parts are twisted relative to each other.

8. An HME device according to claim 6, characterised in that the bypass channel is provided by an annular passage between the outside of the HME unit and the inside of the outer housing.

9. An HME device according to claim 6, characterised in that the HME unit has a tapered coupling at one end removably fitted on a tapered fitting in one part of the outer housing.

10. An HME device according to claim 6, characterised in that the selector includes a shutter plate movable in a plane at right angles to the axis of the device between a position where it covers an opening to the HME unit and reveals a bypass opening to a position where it reveals the opening to the HME unit and covers the bypass opening.
